(19) United States
(12) Patent Application Publication
(10) Pub. No.: US 2009/0142317 A1
(43) Pub. Date: Jun. 4, 2009

(54) PROCESS FOR REDUCING EFFECTS OF GRAFT VERSUS HOST DISEASE USING EX VIVO EXPANDED CD4+CD25+ REGULATORY T CELLS

(75) Inventors: Tinghua Cao, Malvern, PA (US); Li Li, Downingtown, PA (US)

Correspondence Address: HISCOCK & BARCLAY, LLP, 2000 HSBC PLAZA, 100 Chestnut Street, ROCHESTER, NY, 14604-2404, US

(73) Assignee: Therakos, Inc.

(21) Appl. No.: 12/325,464

(22) Filed: Dec. 1, 2008

Related U.S. Application Data
(60) Provisional application No. 60/991,301, filed on Nov. 30, 2007.
(60) Provisional application No. 60/992,347, filed on Dec. 5, 2007.

(51) Int. Cl. A61K 35/12

(52) U.S. Cl. 424/93.71

(57) ABSTRACT

Disclosed in this specification is a process for producing ex vivo expanded CD4+CD25+ regulatory T cells. The process includes the steps of extracting a sample that includes peripheral blood mononuclear cells from a human donor. The extracted cells include a certain number of cells which are CD4+CD25+ regulatory T cells. The relative population of the CD4+CD25+ regulatory T cells is enhanced such that the Treg cells constitute the majority of the cells in the sample. Thereafter, the population of the enriched Treg cells, that may include third-party derived Treg cells, is expanded to produce a clinically meaningful population of cells for use in the treatment of GVHD.

### CROSS-REFERENCE TO RELATED APPLICATIONS

This application claims priority to and the benefit of co-pending U.S. provisional patent applications Ser. No. 60/991,301, filed Nov. 30, 2007, and Ser. No. 60/992,347, filed Dec. 5, 2007, which applications are incorporated herein by reference in their entirety.

### FIELD OF THE INVENTION

This invention relates, in one embodiment, to a process for ex vivo expansion of CD4+CD25+ regulatory T cells. The process includes the steps of extracting a sample that includes peripheral blood mononuclear cells from a human donor. The extracted cells include a certain number of cells which are CD4+CD25+ regulatory T cells. The relative population of the CD4+CD25+ regulatory T cells is enriched such that the Treg cells constitute the majority of the cells in the sample. Thereafter, the population of the enriched Treg cells, that may include the Treg cells derived from third-party donors, is expanded to produce a clinically meaningful population of cells for use in the treatment of GVHD.

### BACKGROUND OF THE INVENTION

Allogeneic hematopoietic stem cell transplantation (HSCT) is a potentially curative therapy for hematological malignancies and inherited hematological disorders. One of the major obstacles and life threatening complications in clinical HSCT is graft versus host disease (GVHD), which is the broad attack against host tissues by activated donor T cells. Although low grade graft versus host effects may play an important role in eradicating malignant cells, severe GVHD is the major cause of mortality and morbidity of patients receiving HSCT. The risk of grade II-IV acute GVHD is up to 70% after allogeneic stem cell transplantation. A variety of immunosuppressive agents, such as calcineurin inhibitors and steroids, are widely used to diminish the risk of GVHD, but more than 50% of grade II-IV GVHD patients are refractory to the current therapies. In addition, the use of high dose immunosuppresants impairs the immune reconstitution, and diminishes T-cell mediated graft versus leukemia (GV L) responses. Due to the high level of unsuccessful treatments with convention therapy, alternative treatments for GVHD are desired.

### SUMMARY OF THE INVENTION

The invention comprises, in one form thereof, a process for producing an enriched sample of CD4+CD25+ Treg cells. The cells isolated and expanded in accordance with the teachings of this invention are useful for treating the symptoms of GVHD.

### BRIEF DESCRIPTION OF THE DRAWINGS

The present invention is disclosed with reference to the accompanying drawings, wherein:

FIGS. 1A, 1B and 1C are graphs of the purity of CD4+CD25+ Treg cells before and after purification;

FIGS. 1D, 1E and 1F are graphs of the purity of CD4+CD25+ Treg cells before and after expansion;

FIG. 2 is a depiction of several graphs showing the phenotypic characteristics of the CD4+CD25+ Treg cells;

FIG. 3A and 3B are graphs depicting certain phenotypic changes in the CD4+CD25+ Treg cells after prolonged expansion;

FIG. 4A, 4B and 4C are graphs showing the in vitro suppressive activities of the CD4+CD25+ Treg cells;

FIG. 5 depicts the effects of the Treg cells on DTH-like local inflammation in NOD/SCID mice;

FIG. 6A to 6E illustrate the effects of the Treg cells on NOD/SCID GVHD mouse model; and

FIG. 7A to 7B are graphs showing expanded human Tregs equivalently inhibited both allogeneic CD4+CD25− T effector T cell proliferation and autologous CD4+CD25− T effector T cell proliferation in in vitro suppression assays.

Corresponding reference characters indicate corresponding parts throughout the several views. The examples set out herein illustrate several embodiments of the invention but should not be construed as limiting the scope of the invention in any manner.

### DETAILED DESCRIPTION

In one embodiment, the invention pertains to a process for extracting human CD4+CD25+Treg cells from healthy donors. Treg cells (i.e. regulatory T cells) are cells that suppress the activation of the immune system thus preventing autoimmune diseases. CD4 and CD25 are proteins that may be expressed by certain cells. Thus, Treg cells which are CD4+ and CD25+ are a subset of Treg cells. A raw blood sample, such as lymphocytes or total blood is withdrawn from a donor. The raw extracted material is purified to enrich the relative population of CD4+CD25+Treg cells. The enriched samples are expanded ex vivo to increase the total cell count while maintaining the relative population of CD4+CD25+Treg cells. The resulting cells are administered to a patient and help to prevent GVHD symptoms.

Human peripheral blood units from healthy donors may be purchased from commercial blood blanks or obtained directly from the donors using convention techniques. Peripheral Blood Mononuclear Cells (PBMC) are first isolated from blood samples by density gradient centrifugation with Ficoll Hypaque (Amersham). The CD4+CD25+Tregs are purified from the isolated PBMC using standard isolation kits (e.g. autoMACS using the human CD4+CD25+ regulatory T cell from Miltenyi Biotec, Auburn, Calif.) according to the manufacturer's instructions. For example, CD4+ T cells are first negatively isolated from PBMC by depleting non-CD4 cells with the mixture of monoclonal antibodies against human CBS, CD14, CD 16, CD19, CD36, CD56, CD 123, TCRY/6 and CD235a. Human CD4+CD25+ Tregs are then positively isolated with anti-human CD25 antibody-conjugated microbeads from the enriched CD4+ T cell population. If desired, the purity of the isolated cells may be determined with flow cytometry after purification.

The purified human CD4+CD25+Tregs are activated and expanded ex vivo in commercial cell culture bags (Miltenyi Biotec and LIFECELL, Baxter) or cell culture plates with CD3/CD28 T Cell Expander Dynalbeads (Invitrogen) in the presence of recombinant human IL-2 (rhIL-2, 1000 U/ml, R&D systems). The CD4+CD25+Tregs were cultured in X-VIVO™ 15 medium supplemented with 10% heat inactivated human AB serum (Lonza, MD), L-glutamine, HEPES, sodium pyruvate, penicillin, streptomycin (Gibco). Fresh medium with rhIL-2 were added 2-3 times per week. After 2 weeks, the CD3/CD28 beads were removed from the Tregs, and the expanded Tregs were then rested for 1-2 days in lower IL-2 (50 U/ml) containing medium before in vitro characterization and function analysis. Certain additives, such rapamycin and/or DRB, may be useful to enrich the sample and maintain high purity during the expansion step.

### Example of Ex Vivo Expansion of Human Treg cells

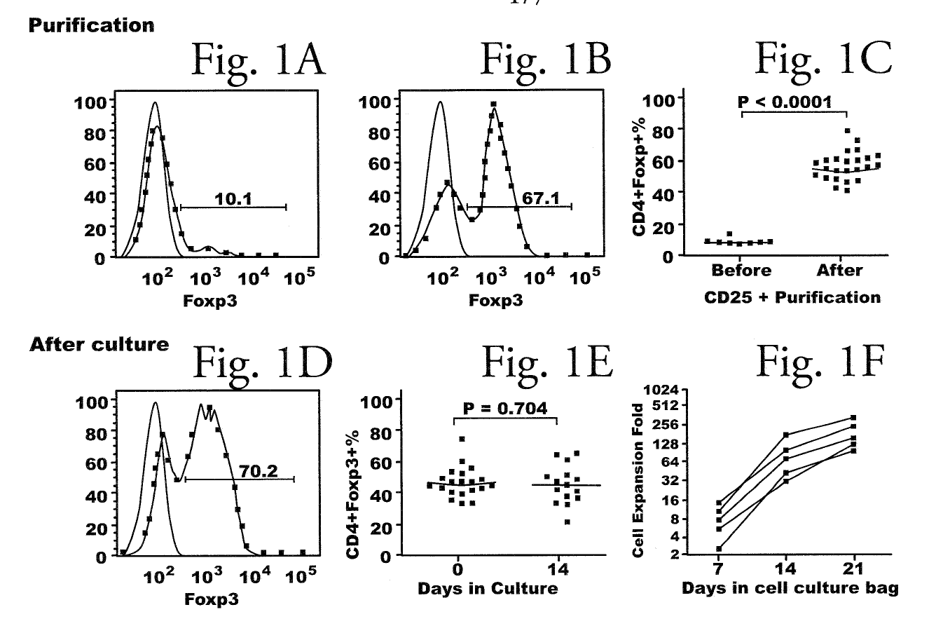

Human CD4+CD25+ Tregs were purified from PBMC from the whole blood units or leukopaks of normal donors (n=16) with autoMACS and human CD4+CD25+ regulatory T cell isolation kits. The purity of isolated CD4+CD25+ Tregs was determined with intracellular Foxp3 staining. CD4 positive cells composed 90% to 98% of those purified cells, of which an average of 55% were Foxp3 positive (range from 40% to 78%) (FIG. 1B, 1C). These results demonstrated that human Tregs can be significantly enriched from PBMC, in which Foxp3+ Tregs constitute only about 1% of the population or 10% of CD4+ T cells (FIG. 1A, 1C). The yield of Tregs was around 0.5% of PBMC. Out of 6 normal donor leukopaks (2-6×10⁹ PBLs) tested, we were able to obtain at least 1×10⁷ Tregs from each donor. The results were also confirmed in large-scale purification using the ClinMACS (Miltenyi Biotec, CA). Advantageously, the population of CD4+CD25+ cells, relative to the overall composition of cells, did not significantly alter when the expansion period was about two weeks. From a functional viewpoint, it is desirable that the expanded population have a composition that is sufficient to maintain the desired biological effect when used therapeutically. In one embodiment, the relative population does not alter more than by about 10%.

The enriched human CD4+CD25+Foxp3+ Tregs were then activated and expanded with CD3/28 T cell expander beads at a 1/3 ratio in the X-VIVO 15™ medium with rhIL2 and 10% of heat-inactivated human male AB serum. In small scale culture plates, the human Tregs were expanded close to 100 fold after two weeks and maintained their purity measured by intracellular Foxp3 staining (n=15, FIG. 1D, 1E). In larger scale cell bag culture (n=10, 4 batches with 100 ml Miltenyi T cell expansion bag and 6 batches with 0.3 to 3 L LIFECELL culture bags), human CD4+CD25+Foxp3+ Tregs were expanded over 100 fold in 2-3 weeks, which was approximately 1 billion cells (FIG. 1F). In the samples that were expanded for fourteen days this represented a fold change of from about 30 to about 300 fold increase. These results demonstrated that clinically relevant human Treg cell numbers could be obtained by large scale ex vivo expansion culture.

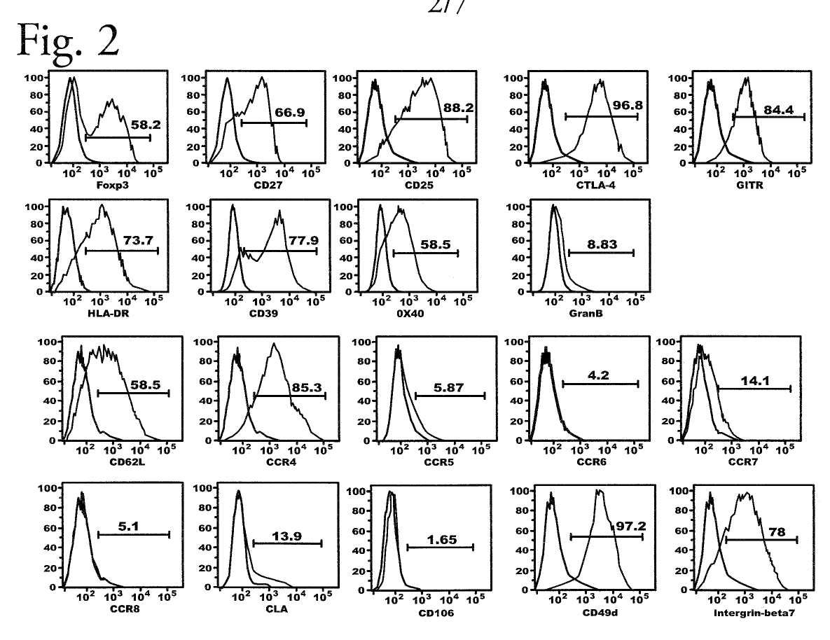

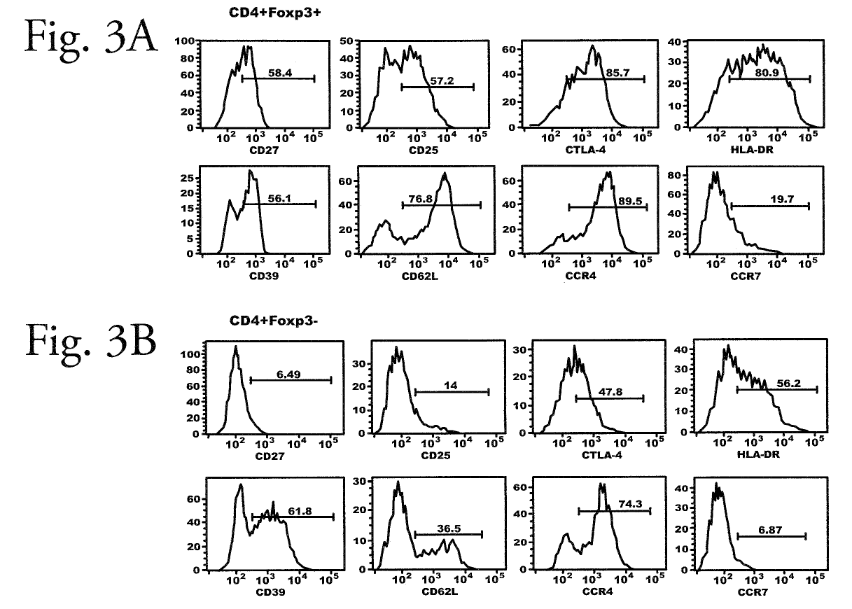

The purity of the week 2 expanded human Tregs was evaluated using intracellular Foxp3 staining as described. Among the 10 cell bag cultures, an average of 57.3% Foxp3 positive cells were obtained (37%, 39%, 45%, 51.8%, 62%, 65%, 68%, 68%, 68% and 70%, respectively). In addition, these cells also showed strong expression of CD27, CD25, CTLA4, GITR, HLA-DR, CD39, CD62L, CCR4, CD49d, intergrinp7, and partial expression of OX40, Granzyme B, CCR7 but negative for CCR5, CCR6, CCR8, CLA, CD106 (FIG. 2). These results suggested that the ex vivo expanded human CD4+CD25+Foxp3+ Tregs retained most of the phenotypic features of human Tregs. The expression of those markers was not significantly different between Foxp3+ and Foxp3− populations in week 2 culture (data not shown). However, in week-3 culture, CD27, CD62L, CD25, and CCR7 were preferentially expressed in the Foxp3+ cells; the Foxp3+ cells also showed higher percentages of CTLA-4, HLA-DR expression than those of the Foxp3− cells (FIG. 3A, 3B).

### Example Showing Ex Vivo Expanded Tregs Maintain Potency In Vitro

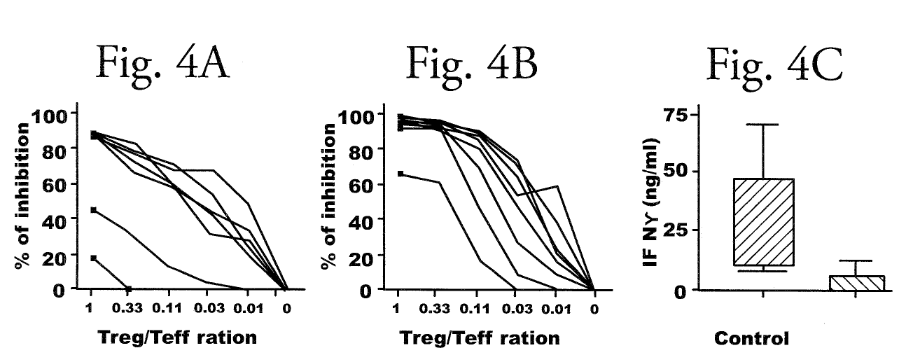

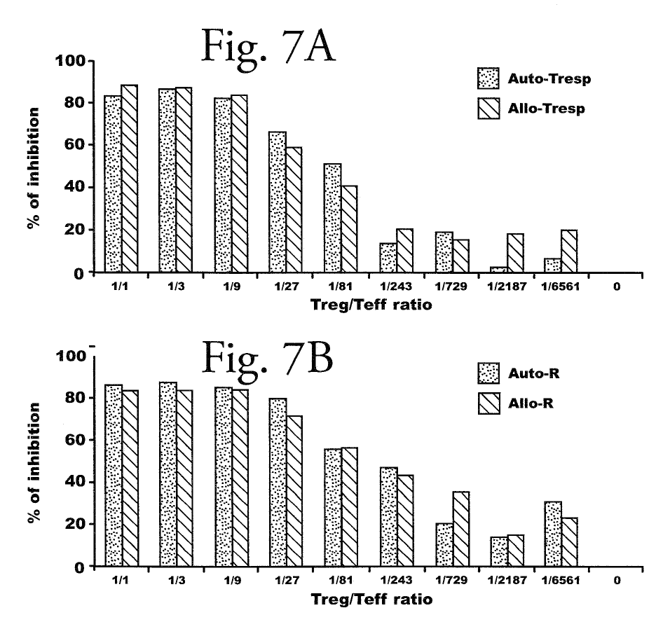

To evaluate the in vitro suppressive function of the ex vivo expanded human CD4+CD25+Foxp3+ Tregs, we generated allogenic dendridic cells (DCs) as antigen-presenting cells and used autologous CD4+CD25− T cells as responder cells. As shown in FIGS. 4A and 4B, ex vivo expanded human CD4+CD25+Foxp3+ Tregs showed potent in vitro suppressive activities in both the MLR and OKT3-induced T cell proliferation assays. In both assays, expanded human Tregs showed a dose dependent inhibition of T cell proliferation (FIG. 4A, B). Most batches of the ex vivo expanded human CD4+CD25+ Foxp3+ Tregs showed more than 50% inhibition of T cell proliferation at the Treg/Teffector ratio of 1/10 to 1/27 in both assays (FIG. 4). In addition, expanded human Tregs inhibited IFNy production in OKT3 assays (FIG. 4C). These results demonstrated that ex vivo expanded human CD4+CD25+Foxp3+Tregs retained strong in vitro suppressive activities. Meanwhile, expanded human Treg cells displayed equal potency to inhibit allogeneic CD4+CD25− T cell proliferation in comparison to autologous CD4+CD25− T cell proliferation (FIG. 7A, 7B).

Human dendritic cells (DCs) were generated from adherent cells or CD14 bead-purified monocytes from PBMC and cultured with RPMI 1640 medium in the presence of 10% FCS, recombinant human GM-CSF (50 ng/ml, R&D systems) and IL-4 (25 ng/ml, R&D systems). Cytokines and medium were changed every other day. On day 5 to 6, DCs were harvested and used for in vitro suppression assays.

The in vitro suppressive activity of ex vivo expanded human Tregs, isolated in accordance with the teachings of this invention, was measured in mixed lymphocyte reaction (MLR) and anti-CD3 antibody induced T cell proliferation assays. In the MLR assay, CD4+CD25− T effector cells (1×10⁵ cells/well) were cultured with allogeneic human dendritic cells (1×10⁴ cells/well) in the 96-well U-bottom plates.

Expanded human Tregs were serially diluted and added into the cultures at different Treg/T effector ratios and cells were cultured for 6 days. At the last 16 hours of culture, ³H-thymidine (1 μCi/well) was added. The plates were harvested and ³H-thymidine incorporation was counted with Topcount (PerkinElmer). Mean counts per minute (cpm) of triplicate cultures and standard deviation were calculated. Percent inhibition of proliferation was calculated as: % inhibition=[(cpm responder cells−cpm responderi Treg)/(cpm responder cells)]×100.

In the anti-human CD3 antibody (OKT3, Ebioscience) induced T cell proliferation assay (OKT3 assay), CD4+CD25− T cells and allogeneic DCs were cultured in 96-well plates in the presence of anti-human CD3 antibody (1 μg/ml, OKT3). Expanded human Tregs were serially diluted and added into the cultures at different Treg/T effector ratios and cells were cultured for 4 days. The readout and the calculation of suppressive activity are the same as those for the MLR assay.

### Example of Xenogeneic GVHD Treatment in NOD/SCID Mice

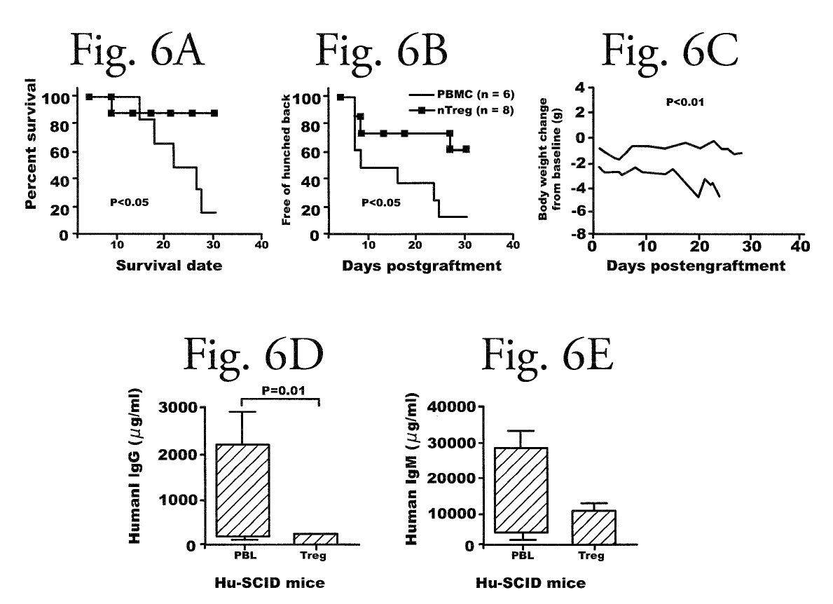

The in vivo activity of ex vivo expanded human CD4+CD25+Foxp3+ Tregs was further evaluated in a xenogeneic GVHD model induced by human PBL in NOD/SCID (non-obese diabetic/Severe combined immunodeficiency) mice. Xenogeneic GVHD was induced by intrasplenic injection of human PBL in the conditioned NOD/SCID mice. As shown in FIGS. 6A to 6C, after transfer of human PBL, the recipient NOD/SCID mice displayed GVHD-like symptoms, e.g. hunched back, diarrhea, and body weight loss, and the mice usually died within 4 weeks.

When co-transferred with PBL into the spleens of the NOD/SCID mice, the ex vivo expanded Tregs significantly enhanced the survival of the NOD/SCID mice (FIG. 6A). Only 1 out of eight mice receiving human PBL together with expanded Tregs died within 1 month; while five out of 6 NOD/SCID mice receiving only human PBL died within 1 month. Meanwhile, ex vivo expanded human CD4+CD25+Foxp3 Tregs also significantly reduced the GVHD symptoms in NOD/SCID mice including hunched back and body weight loss (FIG. 6B, 6C). In addition, expanded human Tregs also inhibited the serum levels of human IgG and IgM in the hu-PBL-NOD/SCID mice. Two weeks post human cell injection, the average concentrations of human IgG and IgM in the sera of hu-PBL-NOD/SCID mice (n 7) with co-transfer of expanded human Tregs were 63.04 pg/ml and 4.548 pg/ml, respectively, in contrast to 1163 pg/ml and 16.398 pg/ml in the hu-PBL-NOD/SCID mice (n 5) without human Tregs (FIG. 6D, 6E). This result suggests that the expanded Treg inhibited human B cell activation and proliferation. Meanwhile, in this study, expanded human Tregs and PBL were derived from different donors, suggesting third-party derived human Tregs prevented GVHD in hu-PBL-NOD/SCID model

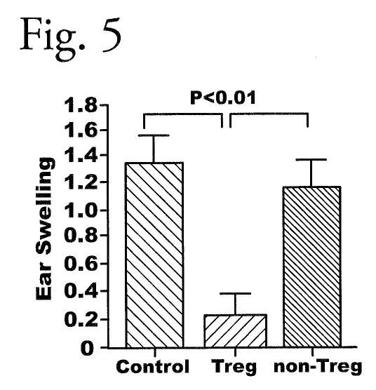

Normal donor PBMC activated with OKT3 were injected subcutaneously into the right ears of the NOD/SCID mice to induce a DTH-like (Delayed type hypersensitivity) local inflammation. The intensity of the DTH was determined by ear thickness measured 24 hrs post cell transfer. As shown in FIG. 5, OKT3-activated normal donor PBMC induced significant DTH compared to the negative control ears, which received the same volume of PBS. When the ex vivo expanded human CD4+CD25+Foxp3+ Tregs (derived from different donor with PBMC) were co-injected with activated normal donor PBMC, at a Treg/PBMC ratio of 1/2, expanded human Tregs significantly inhibited ear swelling induced by the OKT3 activated PBMC (FIG. 5). However, the same amount of non-expanded, non-Treg (CD4+CD25− T cells), when co-injected with the activated PBMC, did not inhibit ear swelling. This result demonstrated that ex vivo expanded human Tregs inhibited an adoptively transferred local DTH response, indicating the expanded Tregs retained their immune suppressive activities in a local tissue environment.

DTH induced by adoptive transfer of human PBMC into NOD/SCID mice DTH response induced by human PBMC in NOD/SCID mice was developed with a modified protocol according to the report by Xu et al (19). Briefly, human PBMC (1×10⁷ cells) were mixed with anti-human CD3 antibody (OKT3, 10 μg per mouse, Ebioscience), with or without ex vivo expanded human CD4+CD25+Foxp3+Tregs (5×10⁶ cells), and were injected subcutaneously (s. c.) in a final volume of 25 μl into the right ears of NOD/SCID mice. The same volume of PBS was injected into the left ears of the same mice as internal controls. Ear swelling, a DTH-like local inflammation induced by the activation of adoptively transferred human PBL, was measured at 24 hours after cell injection with a Series 1010 Starrett calliper. Ear thickness measured before cell injection was used as a baseline control.

One day before the transfer of human cells, the NOD/SCID mice were irradiated (300 rads of gamma irradiation). Mice then received intraperitoneal (i.p.) injection of 20 μl of anti-asialoGMI antibody (Wako Pure Chemical, Osaka, Japan) on days −1, 7, 14, and 21 after the transfer of human cells. Human PBL from healthy normal donors (1×10⁷ cells/per mouse) alone or mixed with ex vivo expanded human CD4+CD25+Foxp3+ Tregs (1×10⁷ cells/per mouse) were then injected into the spleens of the conditioned NOD/SCID mice, or intravenously injected into the conditioned NOD/SCID mice. The detailed procedure of the intrasplenic transplantation of human cells was described previously by Depraetere S et al (J. Immunol. 2001:166:2929-2936). Mouse survival and symptoms of GVHD including hunched back, diarrhea, and body weight were monitored daily. Plasma from the chimeric NOD/SCID mice was collected weekly after cell transfer and human IgG and IgM levels were determined using ELISA kits (Alpha Diagnostic International, TX).

While the invention has been described with reference to certain embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. A process for reducing effects of graft versus host disease using ex vivo expanded CD4+CD25+ regulatory T cells comprising the steps of:
extracting a sample that includes peripheral blood mononuclear cells from a human donor, wherein the peripheral blood mononuclear cells includes CD4+CD25+ regulatory T cells;
enriching the CD4+CD25+regulatory T cells in the sample thus producing enriched CD4+CD25+ regulatory T cells;
expanding the population of the enriched CD4+CD25+ regulatory T cells; and
administering a portion of the expanded CD4+CD25+ regulatory T cells to a human being to treat graft versus host disease.

2. The process as recited in claim 1, wherein the step of enriching the regulatory T cells includes the step of separating the peripheral blood mononuclear cells from the whole blood.

3. The process as recited in claim 2, wherein the step of separating the peripheral blood mononuclear cells includes density gradient centrifugation.

4. The process as recited in claim 1, wherein the step of enriching the CD4+CD25+ regulatory T cells includes the step of negatively isolating CD4+ cells by removing non-CD4 cells using antibodies.

5. The process as recited in claim 4, wherein the step of enriching the CD4+CD25+ regulatory T cells includes the step of positively isolating CD4+CD25+ cells using an anti-human CD25 antibody.

6. The process as recited in claim 1, wherein the step of expanding the population is performed for at least one week, but less than three weeks.

7. The process as recited in claim 6, wherein the step of expanding the population is performed for about two weeks.

8. The process as recited in claim 1, wherein the step of enriching the CD4+CD25+ regulatory T cells produces an enriched sample that is 40% to 78% CD4+CD25+ regulatory T cells relative to the total cell population in the enriched sample.

9. The process as recited in claim 8, wherein, after the step of expanding the population, the sample is 40% to 78% CD4+CD25+ regulatory T cells relative to the total cell population.

10. The process as recited in claim 8, wherein the concentration of the CD4+CD25+ regulatory T cells in the sample, both before and after expansion, are equal within a range of about 10%.

11. The process as recited in claim 1, wherein the enriched CD4+CD25+ regulatory T cells include third-party derived human Treg cells.

12. A process for reducing the effects of graft versus host disease using ex vivo expanded CD4+CD25+ regulatory T cells comprising the steps of:
enriching CD4+CD25+ regulatory T cells in a sample thus producing enriched CD4+CD25+ regulatory T cells;
expanding the population of the separated CD4+CD25+ regulatory T cells, wherein the purity of the CD4+CD25+ regulatory T cells in the sample, both before and after expansion, are equal within a range of about 10%, and
administering a portion of the expanded CD4+CD25+ regulatory T cells to a human being to treat graft versus host disease.

13. The process as recited in claim 12, wherein the step of expanding the population is performed for at least one week, but less than three weeks.

14. The process as recited in claim 13, wherein the step of expanding the population is performed for about two weeks.

15. The process as recited in claim 12, wherein the step of expanding the population is performed for a sufficient period of time to result in a fold change in cell population ranging from not less than 30 fold increase to not greater than 300 fold increase.

16. The process as recited in claim 15, wherein the fold change is not less than 80 fold increase and is not greater than 150 fold increase.

17. The process as recited in claim 12, wherein the enriched CD4+CD25+ regulatory T cells include third-party derived human Treg cells.

18. An ex vivo cellular sample comprising of a plurality of cells, at least 40% of which are CD4+CD25+ regulatory T cells.

19. The cellular sample as recited in claim 18, wherein the CD4+CD25+ regulatory T cells express Foxp3.

20. The cellular sample as recited in claim 19, wherein the CD4+CD25+ regulatory T cells express CD27, CD25, CTLA4, GITR, HLA-DR, CD39, CD62L, CCR4, CD49d, and intergrinp7.

21. The cellular sample as recited in claim 20, wherein the CD4+CD25+ regulatory T cells do not express CCR5, CCR6, CCR8, CLA, and CD106.
